(19) United States
(12) Patent Application Publication
(10) Pub. No.: US 2017/0143898 A1
(43) Pub. Date: May 25, 2017

(54) INFUSION SYSTEM AND METHOD FOR INTEGRITY MONITORING OF AN INFUSION SYSTEM

(71) Applicants: David Grosse-Wentrup, Münster (DE); Uvo Hölscher, Steinfurt (DE)

(72) Inventors: David Grosse-Wentrup, Münster (DE); Uvo Hölscher, Steinfurt (DE)

(21) Appl. No.: 15/300,295

(22) PCT Filed: Mar. 27, 2015
(86) PCT No.: PCT/EP2015/056774

§ 371 Date: Nov. 28, 2016

(30) Foreign Application Priority Data
Apr. 2, 2014 (DE) 10 2014 104 702.8
Nov. 7, 2014 (DE) 10 2014 016 386.5

(51) Int. Cl. A61M 5/168; A61M 5/142; A61M 5/36; A61M 5/14

(52) U.S. Cl. CPC A61M 5/16859; A61M 5/1408; A61M 5/14228; A61M 5/14232; A61M 5/16886; A61M 5/365; A61M 2205/14; A61M 2205/15; A61M 2205/17; A61M 2205/18; A61M 2205/3306; A61M 2205/3334; A61M 2205/3372; A61M 2205/3375; A61M 2205/6081; A61M 2205/6018; A61M 2205/6054; A61M 2205/70

(57) ABSTRACT

An infusion system (1) comprising one or more infusion sources (e.g. pumps and/or reservoirs (2, 3, 4, 9, 10)) and hose lines (5, 11) leading to a patient access (7, 12) for the infusion of a liquid, has a signal generator (A/S), which introduces a pressure signal or an acoustic signal into the liquid, and a sensor (S), which is arranged spaced apart from the signal generator in the infusion system, and an evaluation circuit, which is operatively connected to the sensor in such a way that a sensor signal output by the sensor produces an input signal of the evaluation circuit, said evaluation circuit being operatively connected to a display in such a way that information based on the sensor signals, about the infusion system, such as the flow rate of the liquid, can be displayed. The invention further discloses a method for integrity monitoring of such an infusion system.

The invention relates to an infusion system, as well as a method for monitoring the integrity of an infusion system.

Conventional infusion systems are known in the field. They serve to feed fluids to a patient, for example, into the stomach or into a blood vessel of the patient. They all have an infusion source that serves to transport the fluid, for example, a gravity-fed infusion or an infusion pump, and tubing that is connected to this infusion source, the tubing going from the infusion source to an opening or access point, for example, to a stomach tube, a vein tubule, etc. Fluid is provided within the infusion source and the tubing and is transported by force of gravity or by means of an infusion pump through the tubing to the opening, where the fluid then exits the infusion system.

Elements of the infusion system are regularly replaced for hygienic reasons, for example, every 24 hours, in order to avoid microbial growth. The replacement is done manually and, because of time pressure, errors can occur. For example, the tubing can be connected incorrectly, with the consequence that leaks occur in the infusion system. Stenosis can occur as a result of the patient moving, i.e., the intended flow of the fluid through the infusion system can be negatively impacted or completely interrupted, for example, when the tube line has a kink in it, or when the intended settings are inadvertently incompletely set on stopcocks, multi-port valves, or similar devices, either caused by the patient or when the infusion system is being set up.

Typically, conventional infusion systems encompass not just a single infusion source with a single tube line, but, because multiple medications are being administered. Two or more, for example, four to six medications can be given the patient simultaneously, whereby a dedicated infusion source is provided for each medication. The tube lines that are connected to the sources typically feed into a common tube line within the infusion system, for example, by means of T or Y connectors, with the common tube line feeding into the corresponding opening, for example, into a vein cannula. Due to the possible interactions between the medications it is important with such complex infusion systems, that individual medications, i.e., the individual fluids, from the individual infusion sources are combined in a common subsequent tube line only at certain locations in the infusion system, in order to, for example, avoid an undesirably long stretch in which two medications are together in the tubing at the same time. The medications could exert a negative influence on each other regarding their efficacy, or they could possibly result in flocculation or precipitating out inside the tubing and block the mentioned access point or possibly a filter upstream of the opening.

The previously mentioned problems are of great importance in the area of an intensive care unit or an operating room, because in such situations a large area of the patient is frequently covered up and because of that, the layout of the individual tube lines, as well as the existing branching points in the infusion system are frequently not optically visible, so that a visual monitoring of the infusion system by the medical personnel that are present is frequently not possible or possible only with severe limitations.

It is known to effectively achieve an automatic monitoring of an infusion system by automatically monitoring the function of the infusion system. For example, an increase in pressure in the infusion system can be detected, by providing a pressure sensor in an infusion pump or monitoring the electrical energy that is required to operate an infusion pump is monitored. If, for example, due to stenosis, a higher pressure occurs in the infusion system, so that the infusion pump has to work against this higher pressure, then this condition can be automatically detected, either by directly measuring the prevailing pressure of the fluid in the infusion pump or the force that is exerted on the fluid to be transported, or by detecting a higher than expected energy consumption of the infusion pump. Such automatic monitoring of the infusion system is always problematic then, when very low flow rates occur in the infusion system, namely, when, for example, high-potency medications are given, in which a correspondingly low dosage per time unit is required. In these cases, the corresponding pressure that is caused by a stenosis in the infusion system builds up only over a relatively long period of time, so that the automatic detection and a subsequent possibly required alarm is perhaps possible only relatively late in the process.

Leaks can also occur in the infusion system, for example, at places where the sections of the tube are connected to other components of the infusion system, such as at the infusion source, at branching elements, filters, stopcocks, multi-port valves, or at the mentioned access point, or when the access point itself, for example, becomes detached from the body of the patient. In these cases, a malfunctioning of the infusion system also occurs, because the fluid does not reach the body of the patient as intended, the mentioned automatic monitoring of the infusion system cannot, however, detect and signal an error, because of the lack of a corresponding backpressure.

It is a goal of the invention to improve a conventional infusion system such, that enables an automatic monitoring of the infusion system that makes it possible to monitor the configuration and the condition of the infusion system. Furthermore, it is a goal of the invention to define a method that makes it possible to monitor the integrity of such an infusion system.

This goal is achieved by the infusion system according to claim 1 and by a method according to claim 11. Advantageous embodiments are described in the dependent claims.

The invention suggests, in other words, not a passive analysis of the function of certain components of the infusion system, such as was explained, using the example of the automatic monitoring of an infusion pump, but rather, using a signal generator to actively introduce a signal into the fluid. The signal is transmitted by the fluid and is detected by means of a sensor that is placed at a different location, spaced some distance from the signal generator. The signal can be provided as a single pulse, a series of pulses, or as a longer, possibly continuous signal. Just as an example, the discussion below refers to a pulse, without the present suggestion being restricted to the use of a single, short signal.

The sensor generates a sensor signal and the present suggestion assumes that the signal is influenced by the state of the infusion system as it travels from the signal generator to the sensor. The sensor signals therefore allow conclusions to be drawn regarding the state of the infusion system. The sensor signals are transmitted either directly or after signal processing, i.e., indirectly, to an evaluation circuit that is operatively connected to the sensor and whereby an input signal for the evaluation circuit is generated by the sensor signal, possibly the sensor signal in unchanged form, so that the evaluation circuit can process and evaluate the input signal. Depending on the input signal, the evaluation circuit can activate a display that is operatively connected with the evaluation circuit, so that in the end, depending on the sensor signals, information relating to the infusion system can be displayed.

For example, when an an error is detected, an alarm can be generated, for example, in the form of optical and/or acoustic signals. It can, however, also be the case, that a display also indicates when the infusion system is completely in order. For example, such a display can be constructed as an optical display and the infusion system with its individual components, including the tubing, can be illustrated similar to a diagram showing the rail lines of a train system, so that this illustration of the infusion system, within the context of the present suggestion, is referred to as a “tracks map.” The flow-through rate through the individual rail sections of the tubing can be made clear by correspondingly moved symbols or by color-coded identifiers of the corresponding sections, whereby the color coding possibly provides information about the prevailing flow-through rate in this section of the tubing.

The mentioned signal generator can, for example, be constructed as a pressure generator, so that pressure signals are introduced into the fluid by means of this pressure generator.

A different form of pulses can be provided in the form of acoustic waves, in which the signal generator is constructed as a sound generator, so that the acoustic signals can be introduced into the fluid.

As a further alternative, the signal generator can be constructed as a light generator and, accordingly, light signals be introduced into the fluid.

For purposes of illustration, an infusion pump is hereinafter mentioned as the infusion source, without limiting the present suggestion to this embodiment of an infusion source.

Advantageously, the signal generator can be provided directly in the infusion pump, so that a conventional infusion system is burdened with the least possible additional elements that need to be handled by the medical personnel.

With such integration and when the signal generator is constructed as a pressure generator, the infusion pump can advantageously have a control that can influence the pump rate of the infusion pump as a type of micro-modulation, so that accordingly through, for example, the movement of the plunger on an injection pump provided in the infusion pump can generate a pressure pulse.

Alternatively, the signal generator can be connected to the tubing outside the infusion pump, so that, for example, conventional infusion systems, and particularly conventionally constructed infusion pumps, can also continue to be used without modification. In this case, the signal generator is advantageously provided in an intermediate piece that can be inserted into the tubing so that the fluid can flow through it. This ensures the most direct contact of the signal generator with the fluid, without a wall of the tubing between them, which, depending on the different materials used, could possibly lead to a change in the results or would require regular re-calibration of the infusion system.

As previously mentioned, the infusion system can encompass multiple infusion sources and, thus, the tubing also include multiple tube lines, as well as at least two signal generators, whereby it is advantageous that an individual signal generator be provided for each of the different fluids. The different signal generators are constructed thereby such, that they generate different pulses or signals. In this way, the individual measurement values and sensor signals can be clearly allocated to the respective individual fluids, so that it is possible to achieve particularly clear and far-ranging information as to the state of the infusion system.

Alternatively, the infusion system can have multiple infusion sources and accordingly multiple tube lines, as well as at least one signal generator at a confluence of the lines, i.e., where the tube lines are brought together, so that the equipment requirements of the infusion system are kept to a minimum. More precise monitoring and more differentiated information about the individual components of the infusion system, particularly also about the individual sections of the tubing, are advantageously made possible by providing an individual signal receiver for each infusion source, i.e., for each of the different fluids.

Advantageously, the sensor can serve not only to receive the pulse signals and send out the correlated sensor signals, but it can also be used for generating pulses, if such a sensor is constructed as an actuator sensor element. In this way it is possible, such as, for example, with a bi-directional data transmission, to emit pulses in two different directions within the fluid, so that, for example, the flow velocity of the fluid can be determined automatically, based on the differences in travel time of the corresponding pulse signals.

Different materials can present different obstacles for the pulse signals or can influence the forward transmission in different ways. For that reason, it is advantageous to emit the pulse in the form of a modulated signal, for example, with different frequencies. Thus, for example, if different materials are used for the tubing and the materials attenuate certain frequencies more strongly, they don't negatively influence the forward transmission of the pulse signal, because others of the emitted frequencies can still be transmitted to the sensor with sufficiently strong signal strength.

The same applies for other components that are provided within the infusion system, for example, filters, valves, stopcocks, branching connectors, etc., which, depending on the material and also depending on the settings of the stopcocks and multi-port valves, can present an obstacle for the transmission of the pulse signal. By emitting modulated signals, the probability that at least one part of the signal can pass through the corresponding components of the infusion system and reach the sensor with sufficient signal strength is significantly increased.

In addition, conclusions can be drawn automatically as to the state of the infusion system or its individual components, based on which portions of the signal are weakened or suppressed and which portions of the signal reach the sensor with a significantly greater signal strength, so that the appropriate information can be transmitted to the display by means of the evaluation circuit.

Careful signal analysis can also make possible [sic] the presence of gas bubbles, for example, air bubbles, in the fluid-filled tubing, which are also referred to as infusion tubes. Also the size of these gas bubbles can also be determined in this way.

The present suggestion makes it possible to identify and allocate system components: with wireless components, for example, by receiving modulated, information-coded signals, or by simultaneously actuating (for example, by pressing) control devices (such as keys) at two places in the system, for example, on the one hand pressing an actuator/sensor element, as well as, on the other hand, actuating, for example, a control unit in a central location, the control unit also containing the evaluation circuit.

In an evaluation unit, which typically can be provided as an electronic evaluation circuit, it can be advantageous to gather together two or more or all of the following information:
Information on the state of the components in the infusion system:
   This information is won from the signal evaluation. Different materials of the tubing, different components of the infusion system, such as tubing, valves, stopcocks or multi-port valves, filters, branching connectors, access points, etc., result in characteristic changes of the transmission behavior of the infusion system (1), i.e., the emitted signals result in characteristic echoes because of absorption, transmission, attenuation and/or reflection. Accordingly, statements can be made about the position of a component within the infusion system, the type of component (for example, branching piece, filter, etc.), and its setting (open, closed).
Information from the infusion pumps:
   For example, packaging that contains fluid to be infused can have a machine-readable code printed on it, for example, an RFID tag or a bar code, for example, a QR code. Using a corresponding scanner that is provided in the infusion source, for example, in an infusion pump, information is available as to the type and concentration of the fluid that is carried in the infusion source.
Physician prescription information:
   A hospital has an obligation to maintain documentation on prescriptions and this information can be stored in the form of electronic data in a data storage unit of the hospital, and thus be automatically processed in the evaluation circuit of the infusion system.
Information from pharmaceutical data banks relating to the compatibility of medications:
   This information can also be stored in the form of electronic data in a data storage device of a hospital and can thus also be automatically processed in the evaluation circuit of the infusion system.

An automatic check for completeness, correctness, compatibility, and safety of the infusion system can be implemented by automatically evaluating the previously mentioned available machine-readable data or electronic data. This evaluation can, for example, be done in the evaluation circuit.

The present invention monitors automatically, without involving a user, the state of the fluid system, namely, the infusion tubing and all elements that are connected to them, from the time they are connected to the system over the entire operating time. Because a recognition of the system configuration of the entire system is carried out, a false setup of the system can be immediately recognized, be made known, and thereby be avoided. Furthermore, the various sections of the system, as well as the integrity and function during the entire operation, are continuously monitored. Because of that, it is possible to create a schematic image of the entire infusion system and to display it for the user, just as the rail lines are shown in a graphic display of railroad switch stations.

The system monitoring is based on the detection of the individual system components, including their position in the system and their operating state. A comprehensive image of the entire infusion system can be generated, based on the information from the individual components, such as tubes, tube lengths, valve settings, flow rates and many more, without having a negative effect on the functioning of the system. This knowledge about the system serves the immediate recognition of errors in the setup, function, interconnection, and connection, and to prevent harm to patients.

The collected data are processed by wirelessly connected hardware/software and can be graphically processed in the system to be monitored, as needed or when alarms/errors occur, and provided as filtered data to medical personnel.

Three different technologies are used for the physical monitoring of the system: optical, acoustical, and electrical. They complement each other in their possibilities, but they can also be used singly. They are explained below.

### Acoustic Monitoring

In an acoustic method (sound wave=change in pressure), longer sound signals and/or sound pulses, as well as pressure surges, are introduced into the fluid-carrying system of tubes or into the tubing (i.e., the tube material) itself. Information that allows the identification of the sender/actuator by means of the received signal can be imprinted onto the signals. Disturbances in a fluid propagate as pressure waves, with a pressure wave velocity that is typical for the constellation and the medium.

$$\begin{matrix}Pressure propagation velocity . & Equation 1 \\ {a}_{o} = \frac{{E}_{F}}{ρ} & \end{matrix}$$

α₀=pressure propagation velocity
E_{F}=Elasticity modulus of the fluid
ρ=density of the fluid

These pressure waves propagate in the tubes with the following velocities:

$$a = \frac{{a}_{o}}{\sqrt{1 + \frac{{E}_{F}}{{E}_{R}} + \frac{d}{s}}} \qquad Equation 2$$

α=propagation velocity of the pressure wave in the tube
α₀=velocity of sound in the fluid
E_{F}=Elasticity modulus of the fluid
E_{R}=Elasticity modulus of the tube
d=clear diameter of the tube
s=wall thickness of the tube

The Poisson's ratio of the raw material p goes into the equation as follows:

Pressure wave velocity in the tube including lateral contraction

α=α_{↓}0/√(1+(E_{↓}F*(1−μ^{↑}2)/E_{↓}R+d/s) Equation 3

and is needed in the application, if highly precise results are required.

Monofrequency (for example, sinus) signals, pulses, or, in particular, multi-frequency sweeps and chirps are suitable. Multi-frequency signals are particularly suitable in systems where frequency dependencies aid in characterizing system properties.

The connected components of the system and their properties and the system itself, including the wiring, can then be determined by measuring and evaluating the introduced signals. In addition, fluctuations in pressure generated by the infusion pump or that stem from other sources (for example, the patient) can be evaluated for this purpose.

The pulses are introduced, for example, via their own sound generators that can be attached to the tube, for example, to the infusion tubing, or can be integrated into the infusion pumps. It is also possible to generate the signals by means of micro-modulation of the flow rates of the infusion pumps. Micro-modulation is understood here to mean the short-term change in the rate of infusion, whereby these changes are significantly shorter in duration than the pharmacological half-times of the fastest medications, in order to exclude changes in the pharmacological efficacy of the infusion. The micro-modulation is characterized in that the net infusion rate does not change over a longer period of time, i.e., decreases in the infusion rate are compensated by subsequent increases.

In the first case mentioned, the corresponding signal generators are integrated into the tubing system by means of connector pieces. In the latter case, the pumps themselves are expanded by a signal generating element; as an example, the propulsion mechanics of an injection pump are supplemented with an active element, so that pulses are introduced into the system via the syringe inserted into the injection pump and can be detected. The motor driving the infusion pump can also controlled in a modulated way, so that the corresponding fluctuations in pressure are generated in the tubing system. With peristaltic pumps, a proximally placed (close to the patient) additional peristaltic element can generate these signals or a generator can generate signals in the fluid, passing them through the tubing.

The pressure signals propagate through the system of tubes and are detected by means of electrical actuator/sensor elements at other points in the system, for example, at intersections or end points.

With volumetric pumps, a modified pressure sensor serves to detect the signals.

The signals can be variously constructed, depending on the characteristics of the system:

Each actuator can, based on its location (for example, pump, stopcock, valve, or catheter) have a unique signal characteristic that is clearly coordinated with other locations.

Additionally, special partial segments of the signal can be used in order to transmit information from one pump, such as setting for flow rate, medication, pump ID, etc., via the acoustic system to other pumps or to a common receiver.

Typical algorithms for handling transmission conflicts, such as the Carrier Sense Multiple Access/Collision Avoidance or Carrier Sense Multiple Access/Collision Detection, can be used, so that no disturbance occurs because of interacting transmissions. The actuators can, for example, coordinate the signal output time-wise, when the system is initiated, automatically as part of the self-recognition process, in order to avoid overlapping signals. The pumps can be synchronized and stopped for a brief time, as needed, as soon as one pump sends out a signal; in other words, the pumps provide time slots for sending and receiving among themselves, in which each one actuator sends out a signal and the other actuator/sensor elements listen for the signal response.

The signal response of the signal outputs is taken up at multiple or at all other detection points by the particular actuator/sensor elements. The time difference of the oncoming signals is measured and the difference in travel time of the signal determined as follows:

Signal travel time difference

Δtₛ=t_{S1}−t_{S2} Equation 4
Δt_{S}=travel time difference [ms]
t_{S1}=time of signal detection at Point 1
t_{S2}=time of signal detection at Point 2

The measured and in part weak signals are thereby processed, using signal processing methods. Lock-in amplification can be used for weak signals. In order to obtain an exact measurement of the travel time, the pressure signals have to be analyzed by means of foot-to-foot algorithms (foot-to-foot radius), peak and edge detection, least squares methods, as well as auto-correlation and cross-correlation. This is necessary in order to obtain the most exact determination possible of the travel time and because the pressure signals themselves change on their travel through the line. The above-mentioned methods can be applied simultaneously, in order to obtain even greater accuracy.

The signal parameters that are relevant here are the travel time (this includes the total travel time of the signal through the system, as well as the ratios of the travel times in the individual paths of the system), as well as also the change in the wave form (this includes among others amplitude, frequency, and change over time of the wave form or the period), between each of the individual measuring points or in the echoes.

Also, each actuator/sensor element can receive the different echoes of its own output signal. The combination of the total and partial travel times of the signals in the system allows linear systems of equations to be set up, with which the ratios of the individual lengths of the tubes can be calculated, using Gaussian elimination methods. The result is a definitive wiring diagram of the partial paths. This contains the individual length ratios, interconnections, branchings, and valve settings of connected elements, as well as an estimation of the absolute lengths. The pressure pulse is reflected at the occlusions, for example, at closed stopcocks or at stenoses.

This reflection is recognized by the actuator/sensor element that is sending a signal and the distance to the occlusion determined by means of the signal travel time. Also, at such points of stenosis, depending on the material to be penetrated, signals can be used in frequency ranges that more readily penetrate the corresponding material. Characteristic absorption and transmission of frequency-modulated signals allow in this way statements to be made as to the position and type (for example, stopcock, T-connector, filter) of the occlusion.

If additional properties (for example, E-modulus, inner and outer diameters) of the components used are known, then the actual lengths of the partial paths can be calculated as follows, drawing on Equation 2 and 4:

Length of partial paths

l=Δt_{S}*α Equation 5
l=length of partial path [m]
Δt_{S}=difference in travel time
α=propagation velocity of pressure wave in the line

Even with completely unknown systems, and this can include improperly setup systems with medically irrelevant interconnections, the position of intersections and end points relative each other can be determined from the travel times in this manner or by means of metric multi-dimensional scaling.

Furthermore, the previously mentioned sweeps and chirps can be used on unknown systems to ascertain the system by means of the system response.

Tubing, for example, infusion tubing with generally unknown modulus of elasticity can be characterized by means of a one-time measurement and the following equation, derived from Equation 2, for use in the system:

$$\begin{matrix}E \text{-} Modulus & \\ {E}_{R} = \frac{1}{[ {( ( {a}_{0} * Δ t ) ]}^{2} - 1 ) * ( \frac{s}{d * {E}_{F}} )} & Equation 6\end{matrix}$$

E_{R}=Modulus of elasticity of the line [MPa]
α=velocity of pressure wave propagation in the tube
E_{F}=Modulus of elasticity of the fluid
d=clear diameter of the tube
s=wall thickness of the tube
Δt=difference in travel time

It is also possible to determine the flow rate, based on the velocity of the pressure wave that is changed by the flow and measured by means of bi-directional measurement in the system. In this case, a pressure pulse is send back and forth between each of two communicating actuator/sensor elements. The flow between the elements is determined from the difference in the travel time as follows:

$$\begin{matrix}Determination of flow by means of bi \text{-} directional measurement & Equation 7 \\ \frac{( \frac{1}{2} Δ t * a ) * A}{\frac{1}{2} Δ t} = F & \\ F = Fluss [ \frac{ml}{s} ] & \end{matrix}$$

A=surface of the line
Δt=difference in the signal travel times
α=velocity of pressure wave propagation in the line

In order to refine and verify the measurement, this method can be supplemented with a Doppler frequency measurement of sinus wave signals, whereby an actuator/sensor element sends out a periodic signal that is detected by the other elements.

Subsequently, the partial flows of the individual sections, as well as the total flow rate of the system can be calculated the same way as before by means of Gaussian elimination methods. The flow rates can then be reconciled with the wiring diagram and the specified conditions from the fluid management system.

Given that the network and flow rates in the partial pieces are known, stenoses and leaks can thereby be recognized early, even with low flow rates.

By measuring the transmission behavior of the individual components, the whole system can later be simulated and its properties and function predicted. The entire transmission behavior can then be measured during operation and reconciled with the measured signal travel time. The transmission behavior, as well as the travel time of the signals is a function of the components used, for example, tubing and their properties. Thus, components that are alien to the system can be detected by means of the discrepancy of calculated and measured values for travel time and transmission behavior and their reliability for use in the system checked.

The entire system can be simulated later and its properties and function predicted by measuring the transmission behavior of the individual components.

If systems are then constructed from known elements, then additional statements regarding the system may be made, based on the transmission behavior. This applies to the detection of air bubbles, but also to statements about the fluids used, particularly their density and viscosity. Thus, in the case of infusions, additional support of a check on medication can take place.

Furthermore, with known systems, the system response can be used to measure beyond the limits of the system and into the bordering vascular system of the patient, by means of the needle/catheter. It is particularly important by occlusions at the catheter, that one can ascertain the type of catheter used, based on its echo. In this way, mistaken identifications of peridural and venous catheters, as well as the corresponding incorrect access points, can be recognized.

### Electrical Monitoring

With the electrical method, conductors are attached to the lines and other system elements, for conducting electrical signals. Attaching the conductors is done such, that the electrical connection is ensured when the elements are mechanically connected.

Depending on the complexity of the entire system, the individual elements of the system are provided with analogue and digital components. Thus, the elements of the system can be individually identified. If the individual elements are provided with analogue identification components (resistances, capacitances, inductivities), different statements may be made about the system, depending on the wiring.

If the components are electrically wired in series, the individual strands of the fluid system can be measured and in this way the entire system be recognized. If wired in parallel, the sum of the all of the connected elements can be calculated.

If the system is more complex, digital components, for example, microcontrollers, can be attached to the system elements. This makes it possible to detect each individual element, including its position in the system and also to recognize the individual states of the elements. In this case, these can be the settings of valves, the properties of filters, etc.

In this case, the power is supplied, for example, via the central controller that is attached to one or each of the infusion pumps in the system, via radio (for example, RFID) or induction.

Each of the controllers attached to the elements has an identification number and one or more inputs that are used to read in information about the component, as well as one or more outputs that are sued to forward signals to additional controllers.

### Optical Monitoring

For particular applications, recognition of the connections of the systems can be done by means of light. In this case, depending on the line and the fluid, light is sent through the fluid or through the material of the line. The evaluation is done analogously to the analysis of the acoustic measurement. It is possible in this way to recognize the connected elements and to color code the lines.

A further possibility for the use of light is color-coded marking of different infusion strands, also depending on the infused fluid, or marking defective tubing, be it because of faulty connections or for other reasons, such as the maximal drip duration for tubing that is connected to the patient. Also, internal illumination can make it easier for the user to find a tube or a component that is to be identified or, for example, replaced.

The infusion system according to the invention enables the following advantages that are described only with key words:

By applying pattern recognition to the signals received at the sensor, it is possible to differentiate between information-carrying components of the signal and measurement errors, as well as artifacts. Thus, it is possible to recognize measurement errors caused by bends and sags in the line, coupling vibrations, 50 Hz signal drop-ins, pinching, as well as an inactive line.

When detecting closed T-connectors and other objects that are in or on the tubing, as well as detecting the settings of T-connectors, the attenuation factor of the object, as well as the change in the wave form and phase of the signal caused by the object can be used for identification purposes.

To synchronize the timing of the actuator/sensor modules (A/S), a contact synchronization can be used when installing the system, as well as wireless synchronization methods.

Air bubbles do not have a negative influence on the functioning of the system. With injection pumps, the pressure sensor/actuator can be integrated, for example, into the plunger that presses against the plunger pressure plate of the syringe (vibrating plate).

Peristaltic pumps send oscillations/sound signals based on their mechanics. By modulating the velocity, the signals can be shaped into a clearly detectable form. The peristaltic elements, also by special triggering, can be used for signal detection or signal generation. The ultrasound sensor for air detection can also be so constructed to function as an actuator/sensor (A/S).

Roller pumps are a special type of peristaltic pumps. The rollers of these pumps can be modified to be the actuator.

Additional information can be applied/modulated onto the signals transmitted by the actuators; for example, type of medication and concentration, settings for the rate and pressure limits, pump ID, operating state including alarms, synchronization information including start and stop information. This is done by means of special signal wave forms, sequences, and signal characteristics, such as, for example, wave forms, frequencies (sweeps), pauses.

The properties of the tubing (length, inner diameter, wall diameter, E-Modulus, quality or nature of the tubing) are determined by a change in the signal/transmission behavior over the length of the tubing.

A temperature measurement, for example, at the system components, can be done to compensate for changeable properties, such as, for example, signal line speed.

To measure flow, signal phase shift and travel time can be used. A correction for the different signal paths (through wall and fluid) can thereby be used.

Parallel evaluations of multiple algorithms are compared and compiled to evaluate the signals.

The addition of new elements can be determined once by measuring and then be integrated into the model of the entire system.

Embodiments of the invention are described in greater detail with reference to the purely schematic figures. Shown are:

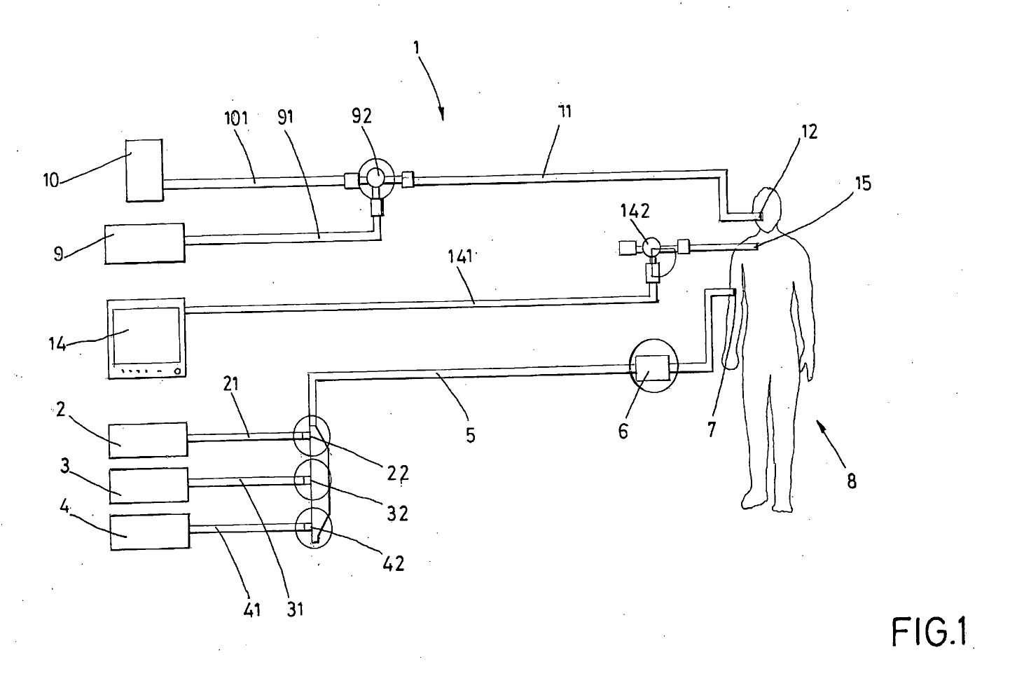

FIG. 1 a representation of an infusion system, referred to as a train lines diagram,

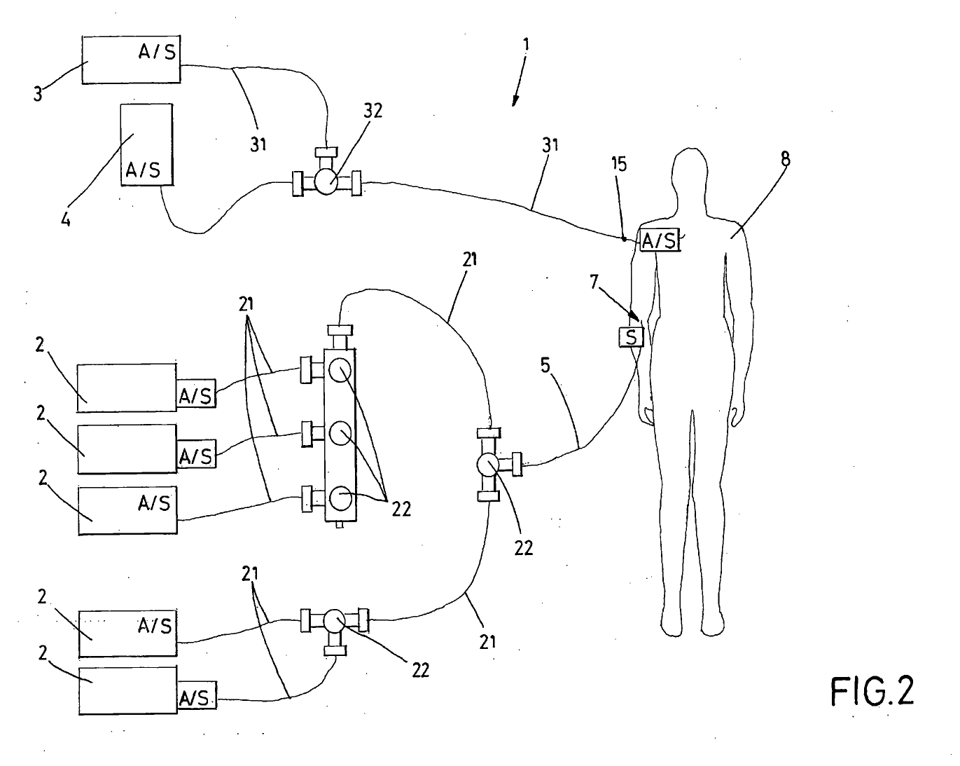

FIG. 2 a schematic representation of a second infusion system, and

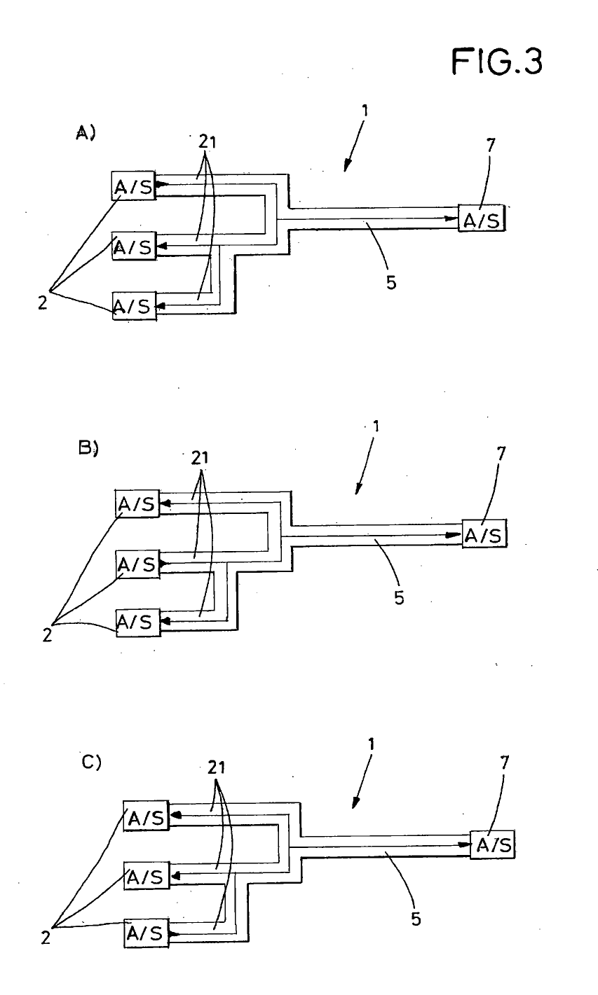

FIG. 3 three different operating states of an infusion system showing signal transmission during a measuring procedure.

FIG. 1 shows an infusion system 1 with three infusion pumps 2, 3, and 4. Infusion tubes 21, 31, 41 lines run from the pumps 2, 3, 4 to multi-path stopcocks 22, 32, and 42. From there, a common tube 5 runs to a filter 6 and on to an access point 7, which is constructed as a vein cannula in the arm vein of a patient 8.

Depending on the switch setting of the multi-path stopcocks 22, 32, and 42, they are marked as “open/permeable in all directions,” “open/permeable in one direction,” or as “closed/impermeable in all directions,” for example, by green rings for open and red rings for closed multi-path stopcocks 22, 32, and/or 42, or by illustration of flow-through openings.

The infusion pumps 2, 3, and 4 serve to transport medications to the patient 8. A further infusion pump 9 transports an additive solution, for example, a saline solution, and a metering pump 10 supplies the patient 8 with nutrition, whereby both the additive solution and the nutrition are carried via tubes 91 and 101 to a multi-path stopcock 92 and from there through a common tube 11 to an access point 12, which is constructed as a stomach probe that is guided through the mouth and throat area of the patient 8.

A patient monitor 14 is also shown in the train lines diagram. This monitor 14 is connected to an access point 15 via a multi-path stopcock 142 and tube 141. The access point 15 is constructed as a central venous catheter and displays and monitors the heart activity of the patient 8.

FIG. 2 shows an infusion system 1 with a plurality of infusion pumps 2, with tube 21 and a plurality of multi-path stopcocks 22 to run to a common tube 5, which serves as a collection line for all fluids of these infusion pumps 2 and carries these fluids to an access point 7 on the patient 8. The access point 7 is constructed as a venous catheter.

A sensor, identified with an “S”, is allocated to the access point 7, whereas an element referred to as actuator/sensor element and identified as “A/S” is provided at each of the individual infusion pumps 2. These actuator/sensor elements serve both as signal generators and sensors.

A further infusion pump 3 is connected via infusion tube 31 to an access point 15 that is constructed as a central venous catheter. Here, too, an actuator/sensor element “A/S” is provided, so that a bi-directional transmission of pulses can occur in this tube 31. Due to the flow of fluid inside the tube 31 in one direction, namely, from the infusion pump 3 to the access point 15, differences in travel time arise between the actuator/sensor elements “A/S”, one being provided at the infusion pump 3 and another provided near the access point 15, so that the flow rate of the fluid can be determined from the difference in travel time.

An additional pump 4 is connected to this same tubing 31 via a multi-path stopcock 32. The infusion pumps 2 and 3 shown in FIG. 2 are constructed as injection pumps, with a syringe plunger that pushes the fluid into the allocated tubing 21 and/or 31. The infusion pump 4, on the other hand, is constructed as a peristaltic pump, just as an example, to make it clear that different types of pumps can be used within the same infusion system 1.

FIG. 3 shows an infusion system 1 in three different states, which are designated A), B), and C). In the infusion system 1, three infusion pumps 2 are provided, each initially connected via its own tube 21 and then via a common tube 5 to an access point 7. Each infusion pump 2 has its own signal generator, as does the access point 7. The signal generator can also be used as a sensor and is therefore referred to as an actuator/sensor element and designated “A/S”. Directional arrows on the actuator/sensor elements “A/S” indicate the direction in which a pulse is sent out through the fluid and/or travels through the fluid.

In state A), the signal generator designated as an actuator of the actuator/sensor elements “A/S” of the upper infusion pump 2 sends out a pulse that travels through the fluid in the tubes 21 and 5 to the sensors of the actuator/sensor elements “A/S” of the other infusion pumps 2 and to the access point 7 and is detected there.

In state B), the actuator of the middle infusion pump 2 sends out a pulse that travels to the sensors of the other infusion pumps 2 and to the access point 7 and can be detected there.

In state C), the actuator of the lower infusion pump 2 sends out a pulse that travels to the sensors of the other infusion pumps 2 and can be detected there.

Because the access point 7 also has an actuator/sensor elements “A/S”, the infusion system 1 may be in a state that is not shown in FIG. 3, a state in which the actuator of the access point 7 sends out a pulse that travels to the sensors of the infusion pumps 2 and can be detected there, for example, in order to determine the flow rates within the framework of a bi-directional pulse transmission.

1. Infusion system (1),
with an infusion source (2, 3, 4, 9, 10),
and a tube (5, 21, 31, 41, 91, 101) that runs from one of the infusion source (2, 3, 4, 9, 10) to one of the access point (7, 12, 15),
and a fluid, which is provided both in the infusion source (2, 3, 4, 9, 10) and in the tube (5, 21, 31, 41, 91, 101) to the access point (7, 12, 15) and is transported by means of the infusion source (2, 3, 4, 9, 10) into the tube (5, 21, 31, 41, 91, 101),
characterized in that,
a signal generator (A/S) is provided that is constructed and arranged to introduce a signal into the fluid,
as well as a sensor (S, A/S) that detects the signal, the sensor being arranged a distance from the signal generator (A/S) in the infusion system (1),
as well as an evaluation circuit that is operatively connected with the sensor (S, A/S) such, that a sensor signal sent out from the sensor (S, A/S) generates an input signal of the evaluation circuit,
and which is operatively connected with a display such, that information about the infusion system (1) that is dependent on the sensor signals is displayable.

2. Infusion system of claim 1,
characterized in that,
the signal generator (A/S) is constructed as a pressure generator such, that a pressure signal is introducible into the fluid by means of the pressure generator.

3. Infusion system of claim 1,
characterized in that,
the signal generator (A/S) is constructed as a sound generator such, that a sound signal is introducible into the fluid or into the tube (5, 21, 31, 41, 91, 101) by means of the sound generator.

4. Infusion system of claim 1,
characterized in that,
the signal generator (A/S) is constructed as a light generator such, that a light signal is introducible into the fluid by means of the light generator.

5. Infusion system of claim 5,
characterized in that,
the signal generator (A/S) is integrated into the infusion source (2, 3, 4, 9, 10).

6. Infusion system 5,
characterized in that,
the infusion source is constructed as the infusion pump (2, 3, 4, 9, 10) and the infusion pump (2, 3, 4, 9, 10) has a control that is constructed such, that the transportation rate of the infusion pump (2, 3, 4, 9, 10) is influenceable in the art of a micro-modulation, generating a sound or pressure pulse.

7. Infusion system of one of the claims 1-4,
characterized in that,
the signal generator (A/S) is connected to the tube (5, 21, 31, 41, 91, 101) outside of the infusion source (2, 3, 4, 9, 10).

8. Infusion system of claim 7,
characterized in that,
the signal generator (A/S) is arranged inside of an intermediate piece, such as a filter, multi-path stopcock (22, 32, 42, 92, 142) or a branching piece, that is inserted in the tube (5, 21, 31, 41, 91, 101).

9. Infusion system of one of the preceding claims,
characterized in that,
the infusion system (1) has multiple infusion sources (2, 3, 4, 9, 10), tubes (5, 21, 31, 41, 91, 101) and at least two signal generators (A/S),
wherein the two signal generators (A/S) are configured to generate different pulse signals.

10. Infusion system of one of the preceding claims,
characterized in that,
the sensor is constructed as an actuator/sensor element (A/S).

11. Method for integrity monitoring of an infusion system (1) constructed according to one of the preceding claims,
wherein a signal is introduced into the fluid or into a tube (5, 21, 31, 41, 91, 101) by means of a signal generator (A/S),
the signal is detected at a location in the infusion system (1) that is some distance from the signal generator (A/S) by means of the sensor (S, A/S), the sensor (S, A/S) outputs a sensor signal that correlates with the detected signal,
an input signal that correlates with the sensor signal is transmitted to the evaluation circuit,
and an information that relates to the infusion system (1) and correlates with the input signal is displayed.

12. The method of claim 11,
characterized in that,
the signal is given off in the form of a pulse or a plurality of pulses in the form of a pulse sequence.

13. The method of claim 11 or 12,
characterized in that,
information is imprinted on the signal.

14. The method of claim 12 or 13,
characterized in that,
the pulse is emitted in the form of a modulated signal having different frequencies.

15. The method of one of the claims 11 to 13,
characterized in that,
an actuator/sensor element (A/S) that serves as a sensor is provided at each of two spaced apart locations of the infusion system (1),
and that bi-directional pulses are generated and evaluated,
and that the flow velocity of the fluid is automatically calculated, based on the different travel times through the fluid flow between the actuator/sensor elements (A/S).

16. The method of claim one of the claims 11 to 15,
characterized in that,
the signal generation and detection is constructed as follows:
an actuator/sensor element (A/S) sends out a pulse-like or periodic signal, the signal is detected by other sensors (S, A/S) provided in the infusion system (1),
the length of the line is subsequently automatically calculated, based on the measurement of the travel time.

17. The method of claim 15,
characterized in that,
the flow as well as the flow velocity in the individual system sections of the infusion system (1) as well as in the entire infusion system (1) is determined by means of an evaluation of the bi-directional measurement.

18. The method of claim 16 and 17,
characterized in that,
the positions and states of the components, as well as the lengths of the lines of the infusion system (1), are determined and graphically represented, based on the signal travel times of the pulses.

19. The method of one of the claims 11 to 18,
characterized in that,
information relating to the infusion system (1) and correlating with the input signal is displayed in the form of an illustration referred to as a track diagram that displays the individual components of the infusion system (1) and information on their states, such as, infusion sources (2, 3, 4, 9, 10), tubes (5, 21, 31, 41, 91, 101), stopcocks, multi-path valves (22, 32, 42, 92, 142), filters, branching pieces, patient probes, access points (7, 12, 15), and that displays leaks and stenoses in the infusion system (1).

20. The method of one of the claims 11 to 18,
characterized in that,
the presence of gas bubbles in the fluid filled tubing is determined, by means of the analysis of the signals received by the sensors (S, A/S).

21. The method of claim 20,
characterized in that,
the size of the gas bubbles is determined.

22. The method of one of the claims 11 to 21,
characterized in that,
the reception of modulated, information-coding signals is provided, for the identification and allocation of wireless system components.

23. The method of one of the claims 11 to 21,
characterized in that,
the simultaneous actuation—such as by pressing—of control devices at two places in the system is done—such as at an actuator/sensor element and at a central location—for the identification and allocation of wireless system components.

24. The method of one of the claims 11 to 23,
characterized in that,
information from the state of the elements in the infusion system (1) as well as additional information is gathered together in the evaluation circuit, the information being information that is available from the infusion sources, information from the prescription information of the physicians,
and/or information from pharmacological data banks on the compatibility of medications.

25. The method of one of the claims 11 to 25,
characterized in that,
an automatic check on the completeness, correctness, compatibility, and safety of the infusion system (1) is carried out.

26. The method of one of the claims 11 to 25,
characterized in that,
a reflection of a signal that is designated an echo is received by means of an actuator/sensor element (A/S), the signal having been output by this actuator/sensor element (A/S) itself and/or from another signal generator, and that, by measuring this echo, the type and position of the reflecting structure is determined,
wherein the characteristic different signal reflections that are generated differently at all changes in diameter, occlusions, and access points of the infusion system (1) are evaluated to determine the structure.

27. The method of one of the claims 11 to 26,
characterized in that,
based on the characteristic change of the transmission behavior of the infusion system (1) (but also absorption, transmission, attenuation, and reflection of modulated signals), statements are made as to the position, type (such as, stopcock, branching piece, filter) and setting (open, closed) of the components used in the infusion system (1).

28. The method of one of the claims 11 to 27,
characterized in that,
initially the transmission behavior of the individual components of the infusion system (1) is measured and later another infusion system (1) is virtually simulated and a statement made as to its properties and function.

29. The method of claim 28,
characterized in that,
the overall transmission behavior of the previously simulated infusion system (1) is measured while in use and reconciled with the calculated signal travel time, wherein, due to the dependency of the transmission behavior as well as the travel time of the signals from the components use, components that are alien to the system are detected from the discrepancy of calculated and measured values for travel time and transmission behavior, such, that their reliability for use in the infusion system (1) is checkable.

30. The method of one of the claims 11 to 29,
characterized in that,
the type of an access point (7) that is connected to a patient (8) is determined by evaluating the way an output signal is reflected.
